(19) United States
(12) Patent Application Publication
(10) Pub. No.: US 2020/0360922 A1
(43) Pub. Date: Nov. 19, 2020

(54) METHOD FOR ASSAYING BIOLOGICAL SAMPLE ON MICROFABRICATED CHIP

(71) Applicant: General Automation Lab Technologies Inc., San Carlos, CA (US)

(72) Inventor: Alexander Hallock, Redwood City, CA (US)

(21) Appl. No.: 16/876,029

(22) Filed: May 16, 2020

Related U.S. Application Data
(60) Provisional application No. 62/849,103, filed on May 16, 2019.

(51) Int. Cl. B01L 3/00; G01N 33/497

(52) U.S. Cl. CPC B01L 3/50855; B01L 2300/0829; G01N 33/497

(57) ABSTRACT

A method of screening for at least one biological entity of interest in a sample using a microfabricated chip having an array of microwells. At least one cell from the sample and a nutrient are loaded into at least one microwell, and a gas permeable membrane is applied to the microfabricated device to retain the at least one cell. The microfabricated device is incubated to grow a plurality of cells. Mass spectrometry is used to detect gas in a sampling region for the at least one microwell. A presence or absence of at least one biological entity of interest in the at least one microwell is determined based on the detection.

### CROSS REFERENCE TO RELATED APPLICATION

This application claims priority to U.S. Provisional Application No. 62/849,103 filed May 16, 2019, the disclosure of which is incorporated by reference herein by its entirety.

### TECHNICAL FIELD

The present disclosure relates generally to innovations in microfabrication, microbiology, analytical chemistry. More specifically, the present disclosure relates to systems methods for high throughput screening and identification of biological entities on microfabricated devices.

### BACKGROUND

Identification and screening microorganisms in microbiomes has been gaining more attention and interest in the chemical, pharmaceutical, agricultural, and other industries as people realize the importance of microbiomes in our health, food production and environment. Useful information can be derived from such efforts, for example, for disease diagnosis and discovery of particular species of microorganisms having certain property of interest or can produce substances of interest.

Recently, various technology platforms utilizing plates or panels containing high density of wells for performing microorganism cultivation and screening have been developed. Due to the extremely small sizes of the wells, and small volume of material involved in each individual well, it is challenging to assay individual wells in-situ. Furthermore, to perform many assays, the contents of the wells need to be accessed and gathered, which disrupts the biological process ongoing in the wells. This often hinders, or even makes it impossible for the well contents to be used for other assays. Although prior to the assay, a replicate of the well contents can be made and transferred to another plate, such a step may require the use of sophisticated equipment and/or techniques, and could introduce errors.

### SUMMARY OF INVENTION

In one aspect, a method of screening for at least one biological entity of interest in a sample using a microfabricated device is provided. The microfabricated device has a top surface defining an array of microwells. The method comprises: loading, into at least one microwell of the array of microwells, at least one cell from the sample and a nutrient; applying a gas permeable membrane to the microfabricated device to retain the at least one cell in the at least one microwell; incubating the microfabricated device at predetermined conditions for a duration of time to grow a plurality of cells from the at least one cell in the at least one microwell; detecting, by mass spectrometry, a gaseous substance in a sampling region exterior and corresponding to the at least one microwell; and determining a presence or absence of at least one biological entity of interest in the at least one microwell based on detection or non-detection of the gaseous substance.

In some embodiments, the detection by mass spectrometry comprises irradiating an area with the sampling region to thereby generate an ionized species from the gaseous substance, if any; and transporting an aliquot of air in the sampling region to a mass analyzer for detection of the existence of any ionized species, to thereby determine the presence of the gaseous substance in the sampling region.

In some embodiments, the at least one microwell includes a plurality of microwells, and detecting the gaseous substance comprises detecting the gaseous substance over an area atop the plurality of microwells.

In some embodiments, the method further comprises correlating the pattern of the detected gaseous substance with the locations of the microwells on the microfabricated chip to thereby determine one or more microwells that have produced the gaseous substance.

The at least one biological entity of interest can comprise a eukaryotic cell, a bacterial cell, etc.

If a biological entity of interest is determined to be present in the at least one microwell, the method can further comprise transferring at least one or some of the plurality of cells from the microwell to a target location.

In some embodiments, the at least one microwell includes a plurality of microwells, and loading the at least one cell comprises loading into each of the plurality of microwells no more than one cell.

In some embodiments, each microwell of the array of microwells has a diameter of about 25 μm to about 500 μm. In some embodiments, the surface density of the array of microwells is at least 750 microwells per cm². In some embodiments, the distance between two neighboring microwells in the array of the microwells of the microfabricated device is less than 500 μm.

In another aspect, a method of screening for at least one biological entity of interest in a sample using a microfabricated device having a top surface defining an array of microwells is provided. The method comprises: loading, into each of a plurality of microwells of the array of microwells, at least one cell from the sample and a nutrient; applying a gas permeable membrane to the microfabricated device to retain the at least one cell loaded in each of the plurality of microwells; incubating the microfabricated device at predetermined conditions for a duration of time to grow a plurality of cells from the at least one cell in each of the plurality of microwells; detecting, by mass spectrometry, a gaseous substance in a sampling region exterior and corresponding to each of the plurality of microwells; and determining a presence or absence of at least one biological entity of interest in each of the plurality of microwells based on detection or non-detection of the gaseous substance. In some embodiments, the microfabricated device is mounted on a stage movable in a plane parallel to a major surface of the microfabricated device, and the detection comprises: positioning an irradiating source to create a focal spot in a first sampling region corresponding to a first microwell to thereby generate an ionized species from the gaseous substance, if any, in the first sampling region; transporting an aliquot of air in the first sampling region to a mass analyzer for detection; laterally moving the stage such that the focal spot of the irradiating source falls in a second sampling region corresponding to a second microwell to thereby generate an ionized species from the gaseous substance, if any, in the second sampling region; and transporting an aliquot of air in the second sampling region to a mass analyzer for detection.

### BRIEF DESCRIPTION OF THE DRAWINGS

FIG. 1 is a perspective view illustrating a microfabricated device or chip in accordance with some embodiments.

FIGS. 2A-2C are top, side, and end views, respectively, illustrating dimensions of microfabricated device or chip in accordance with some embodiments.

FIGS. 3A and 3B are exploded and top views, respectively, illustrating a microfabricated device or chip in accordance with some embodiments.

FIG. 4 is a diagram illustrating a setup for performing an assay on the contents of microwells based on a gas produced in accordance with some embodiments.

FIG. 5 is a diagram illustrating a setup for performing an assay on the contents of microwells based on a gas produced in accordance with some embodiments.

### DETAILED DESCRIPTION

The present disclosure relates generally to systems and methods for isolation, culturing, sampling, and/or screening of biological entities. A microfabricated device (or a “chip”) is used for receiving a sample comprising at least one biological entity (e.g., at least one cell). The term “biological entity” may include, but is not limited to, an organism, a cell, a cell component, a cell product, and a virus, and the term “species” may be used to describe a unit of classification, including, but not limited to, an operational taxonomic unit (OTU), a genotype, a phylotype, a phenotype, an ecotype, a history, a behavior or interaction, a product, a variant, and an evolutionarily significant unit.

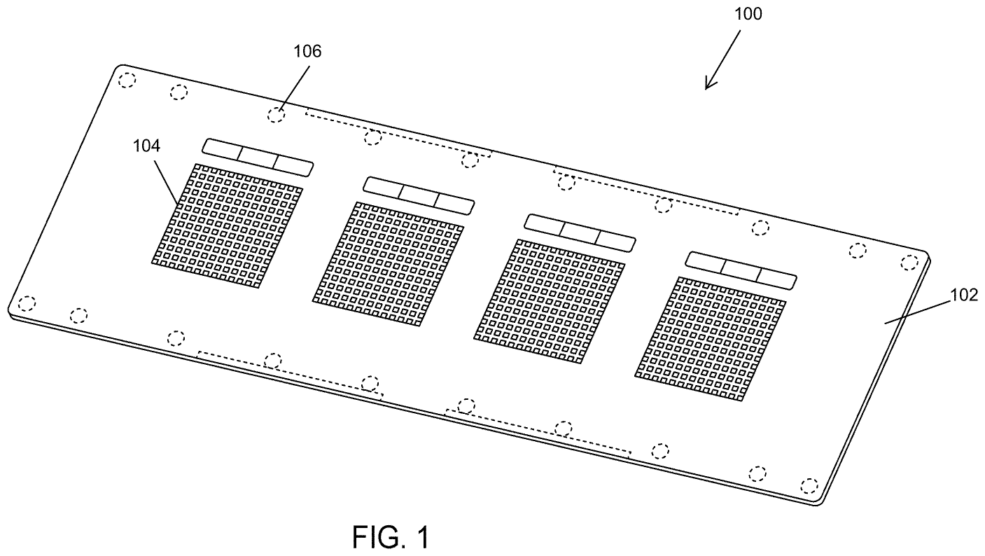

As used herein, a microfabricated device or chip may define a high density array of microwells (or experimental units). For example, a microfabricated chip comprising a “high density” of microwells may include about 150 microwells per cm² to about 160,000 microwells or more per cm² (for example, at least 150 microwells per cm², at least 250 microwells per cm², at least 400 microwells per cm², at least 500 microwells per cm², at least 750 microwells per cm², at least 1,000 microwells per cm², at least 2,500 microwells per cm², at least 5,000 microwells per cm², at least 7,500 microwells per cm², at least 10,000 microwells per cm², at least 50,000 microwells per cm², at least 100,000 microwells per cm², or at least 160,000 microwells per cm²). A substrate of a microfabricated chip may include about or more than 10,000,000 microwells or locations. For example, an array of microwells may include at least 96 locations, at least 1,000 locations, at least 5,000 locations, at least 10,000 locations, at least 50,000 locations, at least 100,000 locations, at least 500,000 locations, at least 1,000,000 locations, at least 5,000,000 locations, or at least 10,000,000 locations. The arrays of microwells may form grid patterns, and be grouped into separate areas or sections. The dimensions of a microwell may range from nanoscopic (e.g., a diameter from about 1 to about 100 nanometers) to microscopic. For example, each microwell may have a diameter of about 1 μm to about 800 μm, a diameter of about 25 μm to about 500 μm, or a diameter of about 30 μm to about 100 μm. A microwell may have a diameter of about or less than 1 μm, about or less than 5μm, about or less than 10 μm, about or less than 25 μm, about or less than 50 μm, about or less than 100 μm, about or less than 200 μm, about or less than 300 μm, about or less than 400 μm, about or less than 500 μm, about or less than 600 μm, about or less than 700 μm, or about or less than 800 μm. In exemplary embodiments, the diameter of the microwells can be about 100 μm or smaller, or 50 μm or smaller. A microwell may have a depth of about 25 μm to about 100 μm, e.g., about 1 μm, about 5 μm, about 10 μm, about 25 μm, about 50 μm, about 100 μm. It can also have greater depth, e.g., about 200 μm, about 300 μm, about 400 μm, about 500 μm. The microfabricated chip can have two major surfaces: a top surface and a bottom surface, where the microwells have openings at the top surface. Each microwell of the microwells may have an opening or cross section having any shape, e.g., round, hexagonal, square, or other shapes. Each microwell may include sidewalls. For microwells that are not round in their openings or cross sections, the diameter of the microwells described herein refer to the effective diameter of a circular shape having an equivalent area. For example, for a square shaped microwell having side lengths of 10×10 microns, a circle having an equivalent area (100 square microns) has a diameter of 11.3 microns. Each microwell may include a sidewall or sidewalls. The sidewalls may have a cross-sectional profile that is straight, oblique, and/or curved. Each microwell includes a bottom which can be flat, round, or of other shapes. The microfabricated chip (with the microwells thereon) may be manufactured from a polymer, e.g., a cyclic olefin polymer, via precision injection molding or some other process such as embossing. The chip may have a substantially planar major surface. FIG. 1 shows a schematic depiction of a microfabricated chip, whose edges are generally parallel to the directions of the rows and the columns of the microwells on the chip.

The high density microwells on the microfabricated chip can be used to conduct various experiments, such as growth or cultivation or screening of various species of bacteria and other microorganisms (or microbes) such as aerobic, anaerobic, and/or facultative aerobic microorganisms. The microwells may be used to conduct experiments with eukaryotic cells such as mammalian cells. Also, the microwells can be used to conduct various genomic or proteomic experiments, and may contain cell products or components, or other biological substances or entities, such as a cell surface (e.g., a cell membrane or wall), a metabolite, a vitamin, a hormone, a neurotransmitter, an antibody, an amino acid, an enzyme, a protein, a saccharide, ATP, a lipid, a nucleoside, a nucleotide, a nucleic acid (e.g., DNA or RNA), etc.

A cell may be Archaea, Bacteria, or Eukaryota (e.g., fungi). For example, a cell may be a microorganism, such as an aerobic, anaerobic, or facultative aerobic microorganisms. A virus may be a bacteriophage. Other cell components/products may include, but are not limited to, proteins, amino acids, enzymes, saccharides, adenosine triphosphate (ATP), lipids, nucleic acids (e.g., DNA and RNA), nucleosides, nucleotides, cell membranes/walls, flagella, fimbriae, organelles, metabolites, vitamins, hormones, neurotransmitters, and antibodies.

A nutrient may be defined (e.g., a chemically defined or synthetic medium) or undefined (e.g., a basal or complex medium). A nutrient may include or be a component of a laboratory-formulated and/or a commercially manufactured medium (e.g., a mix of two or more chemicals). A nutrient may include or be a component of a liquid nutrient medium (i.e., a nutrient broth), such as a marine broth, a lysogeny broth (e.g., Luria broth), etc. A nutrient may include or be a component of a liquid medium mixed with agar to form a solid medium and/or a commercially available manufactured agar plate, such as blood agar.

A nutrient may include or be a component of selective media. For example, selective media may be used for the growth of only certain biological entities or only biological entities with certain properties (e.g., antibiotic resistance or synthesis of a certain metabolite). A nutrient may include or be a component of differential media to distinguish one type of biological entity from another type of biological entity or other types of biological entities by using biochemical characteristics in the presence of specific indicator (e.g., neutral red, phenol red, eosin y, or methylene blue).

A nutrient may include or be a component of an extract of or media derived from a natural environment. For example, a nutrient may be derived from an environment natural to a particular type of biological entity, a different environment, or a plurality of environments. The environment may include, but is not limited to, one or more of a biological tissue (e.g., connective, muscle, nervous, epithelial, plant epidermis, vascular, ground, etc.), a biological fluid or other biological product (e.g., amniotic fluid, bile, blood, cerebrospinal fluid, cerumen, exudate, fecal matter, gastric fluid, interstitial fluid, intracellular fluid, lymphatic fluid, milk, mucus, rumen content, saliva, sebum, semen, sweat, urine, vaginal secretion, vomit, etc.), a microbial suspension, air (including, e.g., different gas contents), supercritical carbon dioxide, soil (including, e.g., minerals, organic matter, gases, liquids, organisms, etc.), sediment (e.g., agricultural, marine, etc.), living organic matter (e.g., plants, insects, other small organisms and microorganisms), dead organic matter, forage (e.g., grasses, legumes, silage, crop residue, etc.), a mineral, oil or oil products (e.g., animal, vegetable, petrochemical), water (e.g., naturally-sourced freshwater, drinking water, seawater, etc.), and/or sewage (e.g., sanitary, commercial, industrial, and/or agricultural wastewater and surface runoff).

FIG. 1 is a perspective view illustrating a microfabricated device or chip in accordance with some embodiments. Chip 100 includes a substrate shaped in a microscope slide format with injection-molded features on top surface 102. The features include four separate microwell arrays (or microarrays) 104 as well as ejector marks 106. The microwells in each microarray are arranged in a grid pattern with well-free margins around the edges of chip 100 and between microarrays 104.

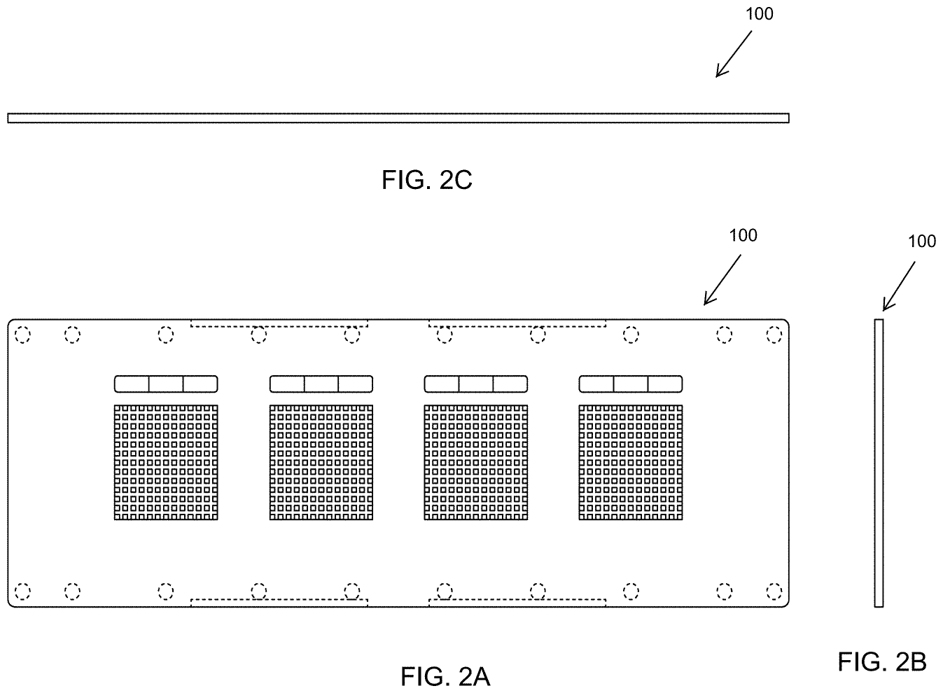

FIGS. 2A-2C are top, side, and end views, respectively, illustrating dimensions of chip 100 in accordance with some embodiments. In FIG. 2A, the top of chip 100 is approximately 25.5 mm by 75.5 mm. In FIG. 2B, the end of chip 100 is approximately 25.5 mm by 0.8 mm. In FIG. 2C, the side of chip 100 is approximately 75.5 mm by 0.8 mm.

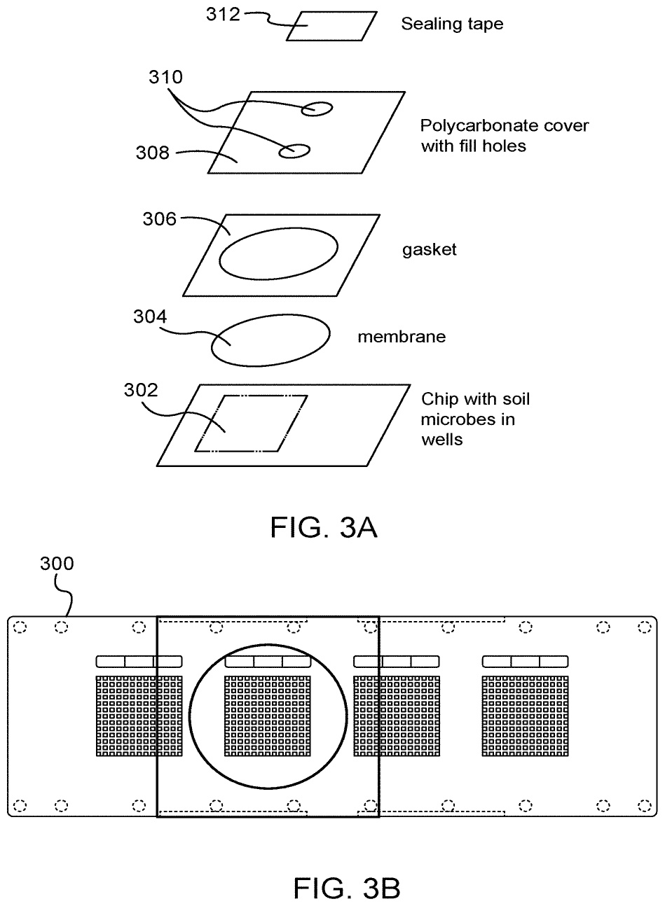

After a sample is loaded on a microfabricated device, a membrane may be applied to at least a portion of a microfabricated device. FIG. 3A is an exploded diagram of the microfabricated device 300 shown from a top view in FIG. 3B in accordance with some embodiments. Device 300 includes a chip with an array of wells 302 holding, for example, soil microbes. A membrane 304 is placed on top of the array of wells 302. A gasket 306 is placed on top of the membrane 304. A polycarbonate cover 308 with fill holes 310 is placed on top of the gasket 306. Finally, sealing tape 312 is applied to the cover 308.

A membrane may cover at least a portion of a microfabricated device including one or more experimental units, wells, or microwells. For example, after a sample is loaded on a microfabricated device, at least one membrane may be applied to at least one microwell of a high density array of microwells. A plurality of membranes may be applied to a plurality of portions of a microfabricated device. For example, separate membranes may be applied to separate subsections of a high density array of microwells.

A membrane may be connected, attached, partially attached, affixed, sealed, and/or partially sealed to a microfabricated device to retain at least one biological entity in the at least one microwell of the high density array of microwells. For example, a membrane may be reversibly affixed to a microfabricated device using lamination. A membrane may be punctured, peeled back, detached, partially detached, removed, and/or partially removed to access at least one biological entity in the at least one microwell of the high density array of microwells.

A membrane may be impermeable, semi-permeable, selectively permeable, differentially permeable, and/or partially permeable to allow diffusion of at least one nutrient into the at least one microwell of a high density array of microwells. For example, a membrane may include a natural material and/or a synthetic material. A membrane may include a hydrogel layer and/or filter paper. In some embodiments, a membrane is selected with a pore size small enough to retain at least some or all of the cells in a microwell. For mammalian cells, the pore size may be a few microns and still retain the cells. However, in some embodiments, the pore size may be less than or equal to about 0.2 μm, such as 0.1 μm. An impermeable membrane has a pore size approaching zero. It is understood that the membrane may have a complex structure that may or may not have defined pore sizes

In one aspect of the present invention, there is provided a nondestructive, spatially-sensitive assay of a biological entity of interest of the samples loaded in the microfabricated chip based on the detection of a gaseous substance (or substances) produced in individual wells by mass spectrometry. The assay can be performed in a highly parallelized manner, enabling fast screening of very large numbers of microbes (e.g. microbiomes) in a single experiment. Compared with alternative methods for assaying contents of microwells, which may involve adding reagents into the well contents, and therefore cause disruption of the normal cell growth/proliferation or even destroy the cells in the microwells (a lot of reagents are harmful or toxic to the cells), the method of the present disclosure separates the site of detection (in the cover film atop of the microwell) from the well contents, therefore does not interfere with cell growth or proliferation in the microwells. This allows the well contents to be available for further assays or tests.

In some embodiments, a method of screening for at least one biological entity of interest in a sample using a microfabricated device is provided. At least one cell from the sample and a nutrient are loaded into at least one microwell of the array of microwells. A gas permeable membrane is applied to the microfabricated device to retain the at least one cell in the at least one microwell. The microfabricated device is incubated at predetermined conditions for a duration of time to grow a plurality of cells from the at least one cell in the at least one microwell. Mass spectrometry is used to detect gas in a sampling region for the at least one microwell. This MS detection can be performed during the incubation or after incubation, and can be done at a single time point, a multiple time points over a time course, or continuously over a period of time. A presence or absence of at least one biological entity of interest, e.g., a eukaryotic cell or a bacterial cell, in the at least one microwell is then determined based on the detection.

The microwell array on the microfabricated chip of the present disclosure is suited for imaging by mass spectrometry. The microwells can be constructed with desired pattern with well-defined parameters (e.g., overall layout, dimension of individual wells, inter-well spacing, etc.) which provide a clear way to relate chemical signals back to the microwell(s) of origin and the location of the microwells. In some embodiments, the microfabricated chip can be made with similar dimensions as a microscope slide, and can be used by standard equipment associated with mass spectrometry without modification. Individual microwells can be resolved by commercial instruments, which routinely achieve a spot size of 10 microns or smaller.

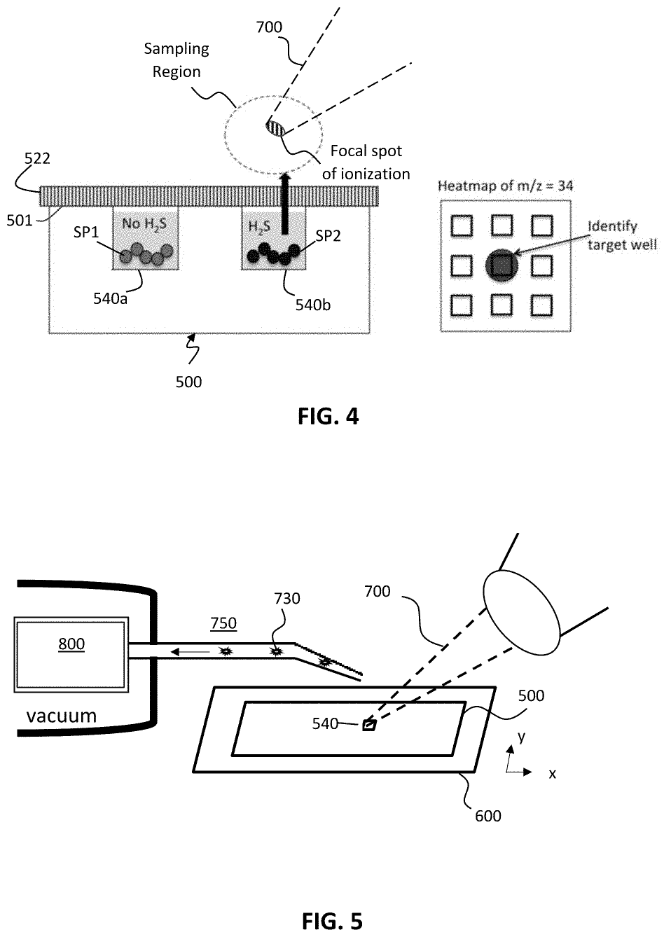

FIG. 4 illustrates an example setup for performing an assay of the contents of microwells based on gases produced from the microwells, which depicts how a mass-derived image can be mapped onto an array of microwells of a microfabricated chip of the present disclosure to determine the microwell(s) of interest and the biological entity of interest contained therein. In this example, microbes that produce hydrogen sulfide (H₂S) are the targets (biological entity of interest). As shown in FIG. 4, the microfabricated chip 500 with microwells 504a and 504b is covered by a gas permeable membrane 522 which allows a gas of interest to come in and/or out of the microwells but retains solid and liquid contents of the microwells. The gas permeable membrane can be applied on and in direct contact with the top surface 501 of the microfabricated device 500 as a seal to retain the contents of the microwells 540a (containing cell species SP1, which do not produce H₂S) and of microwell 540b (containing cells species SP2, which produce H₂S).

While H₂S is used as an example, it is understood that other gas such as oxygen, carbon monoxide, nitric oxide, and ammonia, may be produced in the metabolic activities during the lifecycles of cells (growth, division, multiplication, death), or the reactions or interactions of cells with the micro-environment they are in, e.g., the nutrient loaded in the microwells. For example, sulfate reducing bacteria (SRB) such as Desulfovibrio can react to sulfate contained in a nutrient, producing H₂S as a product which can permeate through the membrane and be detected by mass spectrometry.

It is understood that different gases would have different molecular sizes and affinity to different membrane materials, and their transport rates or behavior through the membrane may also depend on other microstructural parameters of the membrane (which in turn may depend on how the membrane is manufactured, e.g., the cast and drying process, how the pores are created inside the membrane, etc.). The overall gas permeability of the membrane with respect to any particular gas also depends on the thickness of the membrane. The particular membrane (e.g., with specific permeability, thickness and other parameters) selected for any assay can be determined based on the identity and amount of the gas compounds expected to be produced by the microwell contents. In some examples, the gas permeable membrane can be polyurethane sealing membrane with FDA-approved acrylic adhesive commonly used to seal multi-well plates.

The gas permeable membrane forms a tight seal over the microwells. In typical cases, the thickness of the membrane material (e.g., a few microns) will make gas migration from one microwell to another by diffusion through the membrane negligible. In some embodiments, the gas permeable membrane can have a microstructure that enables an anisotropic diffusion of the gaseous substance. The microstructure of the membrane can be such that the gaseous substances can diffuse more rapidly along the thickness direction of the membrane (in other words, perpendicular to the top surface 501 of the microfabricated chip 500) than along its transverse direction (or the direction parallel to the top surface 501). As an example, the gas permeable membrane can have a plurality through channels that are substantially normal to the major surface of membrane. In such a structure, gas diffusion will be predominantly along the direction normal to the membrane surface.

In the example shown in FIG. 4, the mass spectrometer is set to detect a mass/charge ratio of 34 and the microfabricated chip is imaged. In microwells (e.g., 540b) where H₂S is being produced, the gas permeates through the membrane 522 and into the sampling region corresponding to but exterior to the microwells (the space on the other side of the membrane opposing the microwells from which the gas is generated). In some embodiments, the sampling region can have similar cross dimension and/or volume as that of the microwells, e.g., about 10-100 μm in diameter, and is in the vicinity of the corresponding microwells, e.g., having a vertical distance of 10 μm to about 100 μm, e.g., about 10 to about 50 μm, or about 10 μm to about 30 μm, from the membrane above the corresponding microwells. The position and size of the sampling region can be limited by the ionization source used and the focal spot size thereof. While it is preferable to position the focal spot of the ionization energy as close as possible to the membrane to obtain good detection sensitivity, one should avoid unintentionally ionizing the membrane. H₂S molecules can be collected from the sampling region and transported to an ionization chamber for ionization, and the ionized gas molecules are then sent to an MS analyzer for identification/confirmation. Alternatively, the gas can be ionized (at least some of the gas molecules) at the sampling region by a suitable irradiation source, such as a laser, e-beam, plasma, or other means (see FIG. 5 and description) which generates ions from the gas, and the ionized gas is collected and sent for detection by the MS analyzer. The heatmap of m/z=34 (molecular weight of H₂S) can be correlated with a light image of the microfabricated chip to determine which wells produced the H₂S gas. The contents of the microwell(s) identified in this manner can then be sampled, picked or otherwise retrieved for further analysis.

FIG. 5 illustrates a schematic setup for performing an assay by mass spectrometry of the contents of microwells based on a gas (or gases) produced from the microwells. A source of irradiation for ionization, e.g., a focused laser beam 700 (or other beam such as ion beam or e-beam) can be used to create ions from the escaped gas in the sampling region above a microwell 540 (from which a gas has escaped). A small focal spot of the ionization, e.g., a size of 5-10 microns in diameter, in the sampling region above the microfabricated chip (illustrated more clearly in FIG. 4) can be typical, which is smaller than the usual dimension of the microwells and inter-well distances, can enable sufficient spatial resolution in terms of determining from which of the microwells the gas has escaped.

The focal spot of the laser or the ion beam or e-beam can be in the sampling region as illustrated in FIG. 4, i.e., above and apart from the membrane. The focal spot can be moved relative to different microwells on the microfabricated chip so as to generate a map containing mass-to-charge ratio information about the ion species in the sampling region above the membrane. To accomplish this end, the microfabricated chip 500 can be mounted on a stage 600 which can be motorized to move laterally in the x-y plane (parallel to the major surface of the microfabricated device 500) in a controlled manner. This can allow the irradiation source and the associated optical/focusing mechanisms to stay put while enabling a 2-D raster scan over many microwells in a selected area of the microfabricated chip or over the entire chip, and enabling the construction of a picture or map of which microwells are producing what (if any) gas molecules by correlating a pattern of the detected gaseous substance (e.g., the spatial distribution of the presence/absence of the gas substance, and/or the quantity or density of the gas substance over the microfabricated device), with the physical locations of the microwells on the microfabricated chip. The dwell time over each scanned microwell can be dependent upon the strength of the ionization source, the concentration of molecules to be ionized, how easily they are ionized, and what mass range is being queried by the spectrometer. Small, volatile molecules are generally easy to ionize. Adequate signal can be captured in small time intervals, such as one second, of integration over each microwell. Signals over an extended period of time (e.g., minutes or longer) can be integrated to show accumulative effect, as the technique is not destructive to the cells.

More complex laser-based methods can be used. For example, muti-photon ionization, particularly with crossed beams, can be used to restrict the probe volume (down to a spot size of a few cubic microns). Matrix-assisted laser desorption/ionization mass spectrometry (MALDI MS) or matrix free laser desorption mass spectrometry (LDS) can also be used to generate images of the array of microwells (based on the detected gas in the detection area outside the corresponding microwells) in one or more mass-to-charge (m/z) values, providing the capability of mapping specific gas molecules emerged from the microwells to two-dimensional coordinates of these microwells. For example, the matrix in the MALDI MS can be a thin coating applied on the membrane while keeping the gas permeability of the membrane. Alternatively, the laser can be tuned to be in resonance with the membrane material so that the additional matrix coating is not needed. In such instances, the sampling region can be on the outer surface of the membrane (part of the membrane, not off the membrane). Other ionization methods such as plasma or electron guns have even more spatial resolution, where the ionizing beams can be directed down to tens of nanometers.

As shown in FIG. 5, the ions 730 created by the laser or ion beam are collected by capillary tube 750, and mass/charge ratios of the ions can be measured with a mass spectrometer analyzer (or mass analyzer) 800 to determine the chemical composition of the gas produced. The distal tip of the capillary tube can be placed in the sampling region or slightly away from the sampling region, to transport aliquots of air possibly containing the ionized species of interest to the mass analyzer. In any event, one should be careful not to allow any part of the capillary tube to be positioned such that it could be ionized by the ionizing irradiation, which would severely interfere with the intended detection of the gas. The microfabricated chip with the loaded cells is placed at atmospheric pressure and not in vacuum in order not to damage the cells. But the ions need to be transported to an area under vacuum where the spectrometer can run. This can be done simply by means of a capillary tube drawing aliquots of air in atmosphere and continuously feeding it into the mass analyzer. This draw could be pulsed and timed along with the ionization laser (or other ionization source) to read signals only when the scanning stage has a microwell positioned in the right place. The pulsing of the capillary tube could also allow for the instrument to sit until it reads zero to ensure there is no contamination from one read to the next. Suitable software can be used to feed the mass analyzer a continuous stream and deconvolute after data collection. Flow or feed rate of the ions to the mass analyzer can be controlled to allow for sufficient sampling of the ions created. For example, if the ionization volume (for a simple one laser setup) is about 100 cubic microns, the capillary tube can be configured to draw at least 100 cubic microns of air per second (and preferably far more) in order to keep up with the ions being produced.

Based on gas detected (or non-detection) by the mass spectrometer and the location of the detection relative to the microfabricated chip, the microwell or microwells producing the gas can be determined, and the presence or absence of a biological entity of interest in such microwell or microwells can be determined. If a biological entity of interest is determined to be present in such microwell(s), at least parts of the contents of such microwell(s) (including some of the plurality of cells) can be further sampled or transferred to a target location for further growth, cultivation, and/or analysis.

The microfabricated chip can be loaded with a sample containing a plurality of species, strains or genera of microorganisms or eukaryotic cells and the cells can be loaded such that each of the plurality of microwells can contain, on average, one cell, two cells, three cells, four cells, five cells, six cells, seven cells, eight cells, and so on. This can be accomplished using a cell sorter or other available techniques. In some embodiments, the cells can be loaded such only a small subset of the plurality of microwells contain cells to make it unlikely any microwell would contain more than one cell. This is to ensure that if a diverse sample is used, e.g., a sample containing a plurality of microbial cells of different species or genera, each microwell will most likely only contain a single species of cells after incubation.

While various embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed.

What is claimed is:

1. A method of screening for at least one biological entity of interest in a sample using a microfabricated device having a top surface defining an array of microwells, the method comprising:
loading, into at least one microwell of the array of microwells, at least one cell from the sample and a nutrient;
applying a gas permeable membrane to the microfabricated device to retain the at least one cell in the at least one microwell;
incubating the microfabricated device at predetermined conditions for a duration of time to grow a plurality of cells from the at least one cell in the at least one microwell;
detecting, by mass spectrometry, a gaseous substance in a sampling region exterior and corresponding to the at least one microwell; and
determining a presence or absence of at least one biological entity of interest in the at least one microwell based on detection or non-detection of the gaseous substance.

2. The method of claim 1, wherein detecting comprises:
irradiating an area with the sampling region to thereby generate an ionized species from the gaseous substance, if any; and
transporting an aliquot of air in the sampling region to a mass analyzer for detection of the existence of any ionized species, to thereby determine the presence of the gaseous substance in the sampling region.

3. The method of claim 1, wherein the at least one microwell includes a plurality of microwells, and detecting the gaseous substance comprises detecting the gaseous substance over an area atop the plurality of microwells.

4. The method of claim 3, further comprising correlating a pattern of the detected gaseous substance with the locations of the microwells on the microfabricated chip to thereby determine one or more microwells that have produced the gaseous substance.

5. The method of claim 1, wherein the sample comprise a plurality of microbial cells of different species or genera.

6. The method of claim 1, wherein the gaseous substance comprises one of hydrogen sulfide, oxygen, carbon monoxide, nitric oxide, and ammonia.

7. The method of claim 1, wherein the at least one biological entity of interest comprises a eukaryotic cell.

8. The method of claim 1, wherein the at least one biological entity of interest comprises bacteria.

9. The method of claim 1, further comprising:
if a biological entity of interest is determined to be present in the at least one microwell, transferring at least some of the plurality of cells after incubation to a target location.

10. The method of claim 1, wherein the at least one microwell includes a plurality of microwells, and wherein loading the at least one cell comprises loading into each of the plurality of microwells no more than one cell.

11. The method of claim 1, wherein each microwell of the array of microwells has a diameter of about 25 μm to about 500 μm.

12. The method of claim 1, wherein the surface density of the array of microwells is at least 750 microwells per cm².

13. The method of claim 1, wherein a distance between two neighboring microwells in the array of the microwells is less than 500 μm.

14. A method of screening for at least one biological entity of interest in a sample using a microfabricated device having a top surface defining an array of microwells, the method comprising:
loading, into each of a plurality of microwells of the array of microwells, at least one cell from the sample and a nutrient;
applying a gas permeable membrane to the microfabricated device to retain the at least one cell loaded in each of the plurality of microwells;
incubating the microfabricated device at predetermined conditions for a duration of time to grow a plurality of cells from the at least one cell in each of the plurality of microwells;
detecting, by mass spectrometry, a gaseous substance in a sampling region exterior and corresponding to each of the plurality of microwells; and
determining a presence or absence of at least one biological entity of interest in each of the plurality of microwells based on detection or non-detection of the gaseous substance.

15. The method of claim 14, wherein the microfabricated device is mounted on a stage movable in a plane parallel to a major surface of the microfabricated device, wherein the detecting comprises:
positioning an irradiating source to create a focal spot in a first sampling region corresponding to a first microwell to thereby generate an ionized species from the gaseous substance, if any, in the first sampling region;
transporting an aliquot of air in the first sampling region to a mass analyzer for detection;
laterally moving the stage such that the focal spot of the irradiating source falls in a second sampling region corresponding to a second microwell to thereby generate an ionized species from the gaseous substance, if any, in the second sampling region; and
transporting an aliquot of air in the second sampling region to a mass analyzer for detection.
